(19) United States
(12) Patent Application Publication
(10) Pub. No.: US 2006/0134009 A1
(43) Pub. Date: Jun. 22, 2006

(54) Low dose corticosteroid powders for inhalation

(76) Inventors: Daniel Deaver, Franklin, MA (US); Michael Lipp, Framingham, MA (US)

Correspondence Address: ELMORE PATENT LAW GROUP, PC, 209 MAIN STREET, N. CHELMSFORD, MA, 01863, US

(21) Appl. No.: 11/302,535

(22) Filed: Dec. 13, 2005

Related U.S. Application Data
(60) Provisional application No. 60/636,751, filed on Dec. 16, 2004.

(51) Int. Cl. A61K 31/573; A61K 9/14; A61L 9/04

(52) U.S. Cl. 424/46; 514/179

(57) ABSTRACT

The invention relates to a method of treating a corticosteroid-responsive condition of the air passage ways and lungs by delivering a corticosteroid to the pulmonary system of a patient, comprising administering a particle mass comprising a corticosteroid from an inhaler characterized in that the corticosteroid is not substantially deposited in the mouth and throat and is not substantially systemically absorbed. The invention also relates to receptacles containing the particle mass and the inhaler for use therein.

### RELATED APPLICATION

This claims the benefit of U.S. Provisional Application No. 60/636,751, filed on Dec. 16, 2004. The entire teaching of the above application is incorporated herein by reference.

### BACKGROUND OF THE INVENTION

Inhalation of aerosol powders from dry powder inhalers (DPI's) is a convenient way of delivering drugs to patients, such as asthmatics. Current DPI's typically make use of small amounts of micronized drug blended with large amounts of carrier particles, such as a lactose carrier, to facilitate efficient delivery of the drug to the lungs. The efficiency and reproducibility of delivery of such blends is dependent on the patient's lung function and can be affected by parameters such as inspiratory flow rate and/or volume. Existing DPI's can be reservoir based, such as those capable of storing and delivering large numbers of doses to patients, as well as receptacle based, such as those utilizing capsules or blisters. Patients that could benefit from drugs delivered via a DPI often times do have compromised or reduced lung function, which can alter, reduce, or delay the efficiency of delivery or therapeutic onset of the drug.

Conditions leading to such compromised lung function include herein “corticosteroid-responsive conditions of the air passage ways and lungs” (C-RCAL) which include those allergic, non-allergic and/or inflammatory diseases of the upper or lower air passages or of the lungs which are treatable by administering corticosteroids such as those described herein. Typical corticosteroid-responsive diseases include asthma, allergic and non-allergic rhinitis as well as non-malignant proliferative and inflammatory diseases of the air passages and lungs. These include asthma (including bronchial asthma, allergic asthma and intrinsic asthma, e.g., late asthma and airway hyper-responsiveness), chronic bronchitis, inflammatory and/or obstructive diseases of the respiratory tract and/or chronic obstructive pulmonary disease (COPD), respiratory tract infection and upper respiratory tract disease. Other factors such as a patient's age (i.e. children or elderly patients), history (i.e. smoking, chemical exposure) and other conditions can also lead to a reduction of inspiratory flow rate and/or volume.

Many patients who are in need of corticosteroids are incapable of breathing in a sufficient amount of the corticosteroid by means of a MDI or a nebulizer without cumbrous equipment, for example, a face mask attached for the duration of the administration which can be about 3 to about 10 minutes or more. When the patient is in respiratory crisis and in need of treatment full cooperation is required. It can be appreciated that in the case of a young child having an asthma attack, the covering of the mouth and nose with a face mask causes agitation and, in extreme cases, panic. It is not uncommon to have an agitated patient tear off the life saving apparatus.

A need exists to treat compromised lung function and efficiently and reproducibly deliver therapeutic agents, in particular, corticosteroids, to the airways and lungs of compromised patients. Such a treatment would optimally utilize low masses of dry particles capable of being delivered via a single breath-activated step, especially at low inspiratory flow rates and/or low inspiratory volumes. Also, a need exists to deliver the mass of such particles from the DPI to the pulmonary system of a compromised patient.

### SUMMARY OF THE INVENTION

The invention relates to a method of treating a corticosteroid-responsive condition of the air passage ways and lungs by delivering a corticosteroid to the pulmonary system of a patient, comprising administering a particle mass comprising a corticosteroid from an inhaler characterized in that the corticosteroid is not substantially deposited in the mouth and throat and is not substantially systemically absorbed. In one embodiment, the method relates to treating a corticosteroid-responsive condition of the air passage ways and lungs by delivering a corticosteroid to the pulmonary system of a compromised patient, comprising administering a particle mass comprising a corticosteroid from an inhaler containing less than 5 milligrams of the mass, wherein at least about 50% of the mass in the receptacle is delivered to the pulmonary system of the patient and wherein the amount of corticosteroid administered is at least about one-half (½) of the dose of the same corticosteroid administered conventionally. The corticosteroid-responsive condition of the air passage ways and lungs (C-RCAL) includes but is not limited to, asthma, allergic rhinitis, non-allergic rhinitis, non-malignant proliferative and inflammatory diseases include asthma, chronic bronchitis, inflammatory diseases of the respiratory tract, obstructive diseases of the respiratory tract, chronic obstructive pulmonary disease (COPD), respiratory tract infection and upper respiratory tract disease.

Suitable corticosteroids include but are not limited to, aldosterone, beclomethasone, betamethasone, budesonide, cloprednol, cortisone, cortivazol, deflazacort, deoxycortone, desonide, dexamethasone, difluorocortolone, fluclorolone, fluorocortisone, flumethasone, flunisolide, fluocinolone, fluocinonide, fluorocortolone, fluorometholone, flurandrenolone, halcinonide, hydrocortisone, meprednisone, methylprednisolone, paramethasone, prednisolone, prednisone and triamcinolone, and their respective pharmaceutically acceptable salts or esters. Particularly suitable corticosteroids include fluticasone, flunisolide, beclomethasone, and preferably budesonide.

The compromised patient who will benefit from the invention preferably has a peak inspiratory flow rate of about 15 liters per minute. In one embodiment, in a single breath-activated step, the compromised patient is administered a particle mass comprising corticosteroid from an inhaler containing less than 5 milligrams, preferably less than 4 milligrams, more preferably less than 3 milligrams of the mass, wherein at least about 50% of the mass in the receptacle is delivered to the pulmonary system of the patient. In a preferred embodiment the particle mass has a tap density of less than about 0.4 g/cm³, preferably less than about 0.1 g/cm³, even more preferably less than about 0.05 g/cm³. The mass mean geometric diameter of the mass emitted from the inhaler is between about 3 microns and 15 microns, preferably between about 3 microns and 10 microns. Also disclosed is a mass mean aerodynamic diameter of the mass emitted from the inhaler is between about 1 and 5 microns, preferably between about 1 and 3 microns. It is preferred that the emitted dose from the inhaler is greater than about 70%, preferably greater than about 80%. The mass consists essentially of particles produced by spray drying.

The methods of the invention are particularly well suited when the patient is in anaphylaxis and/or is asthmatic. The methods are especially useful if the patient is a child.

In one embodiment the invention is a method of treating corticosteroid-responsive condition of the air passage ways and lungs (C-RCAL) by delivering a budesonide to the pulmonary system of a compromised patient, comprising administering a particle mass comprising budesonide from an inhaler containing less than 5 milligrams of the mass, wherein at least about 50% of the mass in the receptacle is delivered to the pulmonary system of the patient and wherein the amount of corticosteroid administered is at least about one-half (½) of the dose of the same corticosteroid administered conventionally. In this embodiment, the preferred particle mass is less than 4 milligrams further comprising a nominal dose of about 100 to about 350 ug of budesonide, preferably about 200 ug. In one particularly useful formulation the particle mass comprises 5/90/5 weight percent of budesonide/leucine/DPPC.

### BRIEF DESCRIPTION OF THE FIGURES

The present invention is described with reference to the accompanying drawings.

FIG. 1 is a MDSC thermogram for a budesonide formulation of the instant invention.

FIG. 2 is an overlay of DSC and TGA thermograms for the raw drug budesonide.

### DETAILED DESCRIPTION

The invention relates to methods of treating “corticosteroid-responsive conditions of the air passage ways and lungs” (C-RCAL). Disclosed are methods of treating especially vulnerable patients by delivering an agent, in particular a corticosteroid, to the pulmonary system of a compromised patient, in a single breath-activated step, comprising administering a particle mass comprising an agent from an inhaler containing less than 5 milligrams of the mass, wherein at least about 50% of the mass in the receptacle is delivered to the pulmonary system of the patient. In particular, the invention relates to the delivery of corticosteroid formulations at reduced doses compared to conventional DPI (lactose blend) and pMDI corticosteroid formulations which are commercially available. Such a dose advantage potentiates improved corticosteroid pulmonary formulations with reduced side effects. At least in part, the dose advantage minimizes, if not avoids, the systemic effects of the corticosteroids. The corticosteroids delivered by the methods of the invention are not blends as are many of the conventional corticosteroid formulations. Further, the methods of the invention improve treatment outcome by minimizing unwanted depositions of powders in the head and neck area of the patient.

As used herein “corticosteroid-responsive conditions of the air passage ways and lungs” (C-RCAL) means those allergic, non-allergic and/or inflammatory diseases of the upper or lower airway passages or of the lungs which are treatable by administering corticosteroids such as those described herein. Typical corticosteroid-responsive diseases include but are not limited to, asthma, allergic and non-allergic rhinitis as well as non-malignant proliferative and inflammatory diseases of the air passageways and lungs such as bronchial asthma, allergic asthma and intrinsic asthma, e.g., late asthma and airway hyper-responsiveness, chronic bronchitis, inflammatory and/or obstructive diseases of the respiratory tract and/or chronic obstructive pulmonary disease (COPD), respiratory tract infection and upper respiratory tract disease.

The term “asthma” as used herein includes any asthmatic condition marked by recurrent attacks of paroxysmal dyspnea (i.e., “reversible obstructive air passage disease”) with wheezing due to spasmodic contraction of the bronchi (so called “bronchospasm”). Asthmatic conditions which may be treated or even prevented in accordance with this invention include allergic asthma and bronchial allergy characterized by manifestations in sensitized persons provoked by a variety of factors including exercise, especially vigorous exercise (“exercise-induced bronchospasm”), irritant particles (pollen, dust, cotton, cat dander) as well as mild to moderate asthma, chronic asthma, severe chronic asthma, severe and unstable asthma, nocturnal asthma, and psychological stresses.

Corticosteroids are selected from the group consisting of aldosterone, beclomethasone, betamethasone, budesonide, cloprednol, cortisone, cortivazol, deflazacort, deoxycortone, desonide, dexamethasone, difluorocortolone, fluclorolone, fluorocortisone, flumethasone, flunisolide, fluocinolone, fluocinonide, fluorocortolone, fluorometholone, flurandrenolone, halcinonide, hydrocortisone, meprednisone, methylprednisolone, paramethasone, prednisolone, prednisone and triamcinolone, and their respective pharmaceutically acceptable salts or esters.

Particularly useful corticosteroids, including fluticasone, flunisolide, beclomethasone, and preferably budesonide, are efficiently delivered to the lungs for treatment of C-RCAL, particularly asthma. A preferred embodiment is the treatment of asthma in children by administering especially budesonide. Efficiencies in delivery of the formulations of the invention to compromised patients would be expected to lead to lower administered doses. For example, dry powders of the invention containing budesonide, for example, less than about 5 mg, such as less than about 4 mg of total powder deliver therapeutically effective amounts of budesonide such as a nominal dose of about 100 to about 400 ug, and preferably about 200 ug of budesonide.

The invention includes efficient delivery at low inspiratory flow rates, such as in a child, asthmatic or otherwise compromised patient, and ease of an overall inhalation maneuver using the an inhalation system such as that disclosed in co-pending patent application U.S. Ser. No. 10/867,375 entitled “Low Dose Pharmaceutical Powders for Inhalation” and in U.S. application Publications disclosing inhalation devices which applications are assigned to Advanced Inhalation Research, Inc. including 2003/0150453, 2004/0011360, 2004/0154618, 2004/0154619 and 2004/0216738. The entire teaching of these and all patents and patent applications in this disclosure are incorporated herein by reference unless otherwise noted.

Since orally-delivered corticosteroids have significant systemic effects on the body, inhaled corticosteroids have been developed to be effective when directly inhaled into the lungs, with less systemic absorption or fewer side effects when used in low doses relative to oral administration. Corticosteroid formulations currently available inside and outside of the U.S. include Flovent Rotadisk (fluticasone propionate, GSK), Pulmicort Turbuhaler (budesonide, Astra-Zeneca), Aerobid (flunisolide, Forest Labs.) and Beclovent (beclomethasone, GSK), among others (see Table 2 for dosage information). The use of inhaled corticosteroids thus has a reduced incidence of systemic side effects compared to oral corticosteroid therapy. However, currently available formulations are not problem-free. The inefficiency of standard DPI and pMDI corticosteroid formulations results in a low efficiency of dosing (typically on the order of 10 to 15% of the inhaled dose reaching the target sites in the lungs), resulting in systemic exposure via deposition in the oropharyngeal cavity and the throat. For example, it is commonly recommended that individuals using inhaled corticosteroids rinse their throats following use in order to avoid sore throats, yeast infections, and hoarseness.

The invention solves the current problem of sore throats, yeast infections and hoarseness which usually accompany conventional inhaled corticosteroid administration (for example, via nebulization and MDIs) by providing efficient delivery to the lungs with minimum head and neck deposition. Further, it should be noted that this deposition from conventional inhaled corticosteroid administration can be swallowed thereby leading to absorption through the stomach and higher systemic exposure. The reduced side effects serve to spare the compromised patient additional challenges to an already weakened immune system. It can be appreciated that when the overall system is no longer as taxed as by conventional therapies, additional benefits not yet listed may accrue to the treated patient.

Applicants have been improving methods of delivering particle masses, in particular, dry particles comprising corticosteroid for pulmonary delivery. Conventional oral, systemic corticosteroid therapy is known to be associated with the potential for significant short and long-term side effects (some of the common side effects are listed in Table 1 below), as well as with the occurrence of “steroid withdrawal” upon cessation of therapy. Adverse steroid withdrawal effects may include muscle aches, joint pains, fatigue, poor appetite, and fever. Individuals coming off corticosteroids may also be at risk for symptoms that were suppressed during corticosteroid treatment such as skin problems (eczema, hives), hay fever and sinus symptoms, and arthritis-like symptoms. Additionally, when corticosteroids are taken as a medication, they can suppress the normal secretion of corticosteroids from the adrenal gland, as a result of inhibiting ACTH secretion from the anterior pituitary gland. If the corticosteroid medication is given over a very long time, with significant systemic exposure, adrenal gland to hypotrophy is possible.

TABLE 1
Common side effects associated with corticosteroid therapy (Taken from
the Asthma Center Website, www.asthmacenter.com).

| | |
|---|---|
| Weight gain | Gastric ulcer |
| Water retention | Thinning of skin |
| Filling out of the face | Increased hair growth in women |
| Osteoporosis (weak bones) | Increased blood sugar |
| High blood pressure | Loss of hair |
| Damage to hip bone | Increased risk of infections |
| Inhibition of growth in children | Increased bruising of skin |
| Muscle weakness | Personality changes |
| Cataracts | Menstrual changes |
| Glaucoma | Depression |

The methods of the invention employ the delivery of an agent, especially corticosteroid formulations, to the pulmonary system of a compromised patient, in a single breath-activated step, comprising administering a particle mass comprising an agent from an inhaler containing less than 5 milligrams of the mass, wherein at least about 50% of the mass in the receptacle is delivered to the pulmonary system of the patient. In particular, the invention relates to the delivery of corticosteroid formulations at reduced doses compared to conventional DPI (lactose blend) and pMDI corticosteroid formulations. Such a dose advantage can potentially allow for improved corticosteroid pulmonary formulations with reduced side effects. In the case of corticosteroids delivered by the methods of the invention, not only are blends unnecessary but also the methods minimize unwanted depositions of powders in the head and neck area of the patient.

Compromised patients include individuals who do not or cannot breathe hard or have a compromised lung function. Examples of such individuals include children, elderly persons, persons suffering from respiratory disease, such as conditions leading to such compromised lung function including asthma, COPD, anaphylaxis, emphysema, and other forms of respiratory distress. Other factors such as a patient's age (i.e. children or elderly patients), history (i.e. smoking, chemical exposure) and other conditions can also lead to a reduction of inspiratory flow rate and/or volume. Other individuals include sleeping individuals and comatose individuals. Preferably, the individuals are vertical during the method. However, it is also possible to practice the method horizontally. Preferably, the corticosteroid, especially budesonide, is administered to children at doses of less than 5 mg, such as less than 4 mg.

Generally, the individual suffering from corticosteroid-responsive condition of the air passage ways and lungs will have a peak inspiratory flow rate (PIFR) of less than about 20 liters per minute and often, much less. In one embodiment, the method is employed in a patient having a PIFR of about 15 liters per minute or less. Alternatively or additionally, the compromised patient has an inspiration volume of less than 2 liters, such as less than 1.5 liters, including less than 1 liter, such as 0.75 liters.

The method is particularly useful in delivering agents that treat the cause of the patient's compromised state, agents include corticosteroids. Particularly useful corticosteroids are selected from the group consisting of aldosterone, beclomethasone, betamethasone, budesonide, cloprednol, cortisone, cortivazol, deflazacort, deoxycortone, desonide, dexamethasone, difluorocortolone, fluclorolone, fluorocortisone, flumethasone, flunisolide, fluocinolone, fluocinonide, fluorocortolone, fluorometholone, flurandrenolone, halcinonide, hydrocortisone, meprednisone, methylprednisolone, paramethasone, prednisolone, prednisone and triamcinolone, and their respective pharmaceutically acceptable salts or esters.

The invention includes the delivery of very small amounts of dry powders comprising corticosteroids which delivery can be achieved independently of PIFR. For example, in the particular application of the delivery of corticosteroids, especially budesonide, very low dose formulations are possible. Indeed, the target dose is as low a dose that is clinically effective and at least up to about one-half of the current standard DPI and pMDI doses. This discovery permits reliable and reproducible dosing for the patient, irrespective of the patient's particular condition and the need to determine the patient's actual flow rate prior to administering the dose, even at very low doses of particle mass. The combination of the selected drug, the selected dosage, the selected condition, and the selected population of patients coupled with the avoidance of selected side effects and the minimization, if not elimination of, systemic delivery creates a unique treatment option.

Thus, in a preferred embodiment, the inhaler, or the receptacle which may be disposed within the inhaler, contains less than 4 milligrams of the particle mass, preferably less than about 3 milligrams. In one embodiment, the mass of particles contains budesonide at a dose of about 100 to about 400 ug, and preferably about 200 ug of budesonide.

The particle mass comprising corticosteroids is highly dispersible and possesses good to excellent deposition in the lung. Examples of preferred particle masses possess a tap density of less than about 0.4 g/cm³, preferably less than about 0.1 g/cm³, such as less than about 0.05 g/cm³. Tap density is a measure of the envelope mass density characterizing a particle. The envelope mass density of particles of a statistically isotropic shape is defined as the mass of the particle divided by the minimum sphere envelope volume within which it can be enclosed.

Preferred particle masses comprising corticosteroids possess a mass mean geometric diameter of the mass, as emitted from the inhaler, between about 1 micron and 20 microns, such as between about 3 and 15 microns, more preferably between about 3 microns and 10 microns. Good deposition to the lung can be achieved with particle masses possessing a preferred mass mean aerodynamic diameter of the mass emitted from the inhaler is between about 1 to 5 microns, such as between about 1 and 3 microns. Preferred particles masses include or consist of spray-dried particles.

Features that can contribute to low tap density, large geometric diameter and low aerodynamic diameter include irregular surface texture and hollow or porous structure. Particularly preferred particles are described in U.S. Pat. Nos. 6,136,295; 5,985,309; 5,874,064; and 5,855,913. U.S. patent application Ser. No. 09/591,307, filed Jun. 9, 2000 entitled “High Efficient Delivery of a Large Therapeutic Mass Aerosol,” and related applications entitled “Highly Efficient Delivery of a Large Therapeutic Mass Aerosol,” published as U.S. Publications 2002/0035993 and 2004/0076589, are also instructive. The entirety of each of the foregoing patents and patent applications is hereby incorporated by reference.

The method of treating the compromised individual relies in part on good to excellent emitted doses of the particle mass. In one embodiment, the emitted dose is at least 50%, preferably at least about 60%, more preferably at least about 70%. In a particularly preferred embodiment, the achieved emitted dose can be greater than about 80% such as at least about 85%.

Suitable inhalers are disclosed in the patent application filed in the United States Patent & Trademark Office on Oct. 10, 2002, having the title “Inhalation Device and Method” by Edwards et al., U.S. patent application Ser. No. 10/268,059. Other inhalers that can be used include those described in PCT publication WO 02/083220, having the title “Inhalation Device and Method,” by Edwards et al. published on Oct. 24, 2002. The contents of the applications are incorporated herein by reference in their entirety, together with their priority documents.

Alternative inhalers which can be used in the method are dry powder inhalers, including capsule loaded inhalers. Examples of suitable dry powder inhalers include but are not limited to the inhalers disclosed in U.S. Pat. Nos. 4,995,385 and 4,069,819, SPINHALER (Fisons, UK), ROTAHALER (GlaxoSmithKline, NC), FLOWCAPS (Hovione, Switzerland), INHALATOR (Boehringer-Ingelhein, Germany), AEROLIZER (Novartis, Switzerland), and the ECLIPSE (Aventis) and blister-based inhalers, such as DISKHALER (GSK, NC), and DISKUS (GSK, NC).

In co-pending U.S. Ser. No. 10/867,375, the teaching of which are incorporated herein by reference, the effectiveness of the low dose powders was compared against other available inhalers such as the Diskhaler, Inhalator, and Eclipse. Another known inhaler not yet commercially available is the C2S inhaler. Such technologies are useful in the present invention.

### EXEMPLIFICATION

### Example 1

Very low dose corticosteroid formulations with higher aerosolization and pulmonary deposition efficiencies provide significantly improved corticosteroid therapies for the treatment of C-RCAL, especially asthma.

TABLE 2
Currently available corticosteroid dosage strengths and pediatric doses.

| Corticosteroid | Available dosage | Recommended pediatric |
|---|---|---|
| formulation | strengths | doses |
| Fluticasone | 44 to 220 ug per inhalation | 88 to 440 ug, 2 × day |
| (Flovent) | | |
| Budesonide | 200 ug per inhalation | 200 to 400 ug, 2 × day |
| (Pulmicort) | | |
| Flunisolide | 250 ug per inhalation | 500 ug, 2 × day |
| (Aerobid) | | |
| Beclomethasone | 40 to 80 ug per inhalation | 40 to 80 ug, 3-4 × day or |
| (Beclovent) | | 160 ug, 2 × day |

Table 3 discloses the selection of target corticosteroids and evaluative criteria for use in the practice of the invention. Below is the target information for budesonide. Additional corticosteroid candidates are examined based on preliminary results for budesonide. Evaluative criteria for determining the success of this feasibility program are displayed in Table 3.

TABLE 3
AIR-corticosteroid formulation targets.

| Property | Target |
|---|---|
| Product dosing frequency | 2X daily dosing |
| Formulation drug load/ | Target is as low a dose as is clinically |
| target dose | effective + at least ½ of current standard |
| | DPI + pMDI doses |
| Formulation excipients | Ideally excipients with good safety profiles |
| | and existing toxicity data. |
| Powder geometric size | VMGD > 5 microns, ρ < 0.4 g/cc |
| and density | |
| Powder fine particle | FPF < 5.6 microns > 60% |
| fraction | |
| Powder physical | Stability to thermal + humidity stresses and |
| stability | to amorphous to crystalline conversions |
| Powder chemical | Target |
| stability | |
| PD response | Equivalent or better than standard DPI + pMDI |
| | therapies at least ½ nominal drug |
| | dose |

### Example 2

Formulation selection and spray-drying of formulations of corticosteroids useful in the practice of the invention are disclosed. Excipients and formulations are screened and selected based on the targets described above. Approximately 2 to 8 initial budesonide (BU) formulations are chosen for spray-drying, with additional formulations developed based on the initial characterization results. Formulations are spray dried to achieve dry particle powder compositions by manipulating various solution and process parameters that affect particle physical properties.

### Example 3

As part of formulation optimization, BU powders produced as described above are analyzed for the following physical properties: geometric size (RODOS/HELOS laser diffraction system), aerodynamic size (Aerosizer system) and tap density (Van Kel tap density analyzer). Optimized powders for physical characteristics and API stability are further analyzed and screened for their aerosolization performance upon emission from an inhaler via: emitted dose (ED), fine particle fraction (FPF) and emitted geometric size (RODOS-IHA), as well as their solid-state properties such as thermal transitions (DSC), water content (Karl Fischer), hygroscopicity (DVS) and morphology (SEM). Lead formulation(s) are selected for further testing based on these results.

### Example 4

Accelerated stability testing is performed to assess the physical and chemical stability of the lead BU formulation(s) resulting from the initial screening process described above. The Lead formulation(s) are exposed to different temperature and relative humidity conditions for periods of time up to 4 weeks. These powders are evaluated at various timepoints for their physical and chemical properties including particle aerodynamic characteristics, physical molecular properties (amorphous to crystalline conversions, etc.) and formulation chemical stability.

### Example 5

Based upon the outcome above, optional development is performed. For example, Applicants perform (i) additional formulation optimization based on the results of the studies described above and the formulation target goals, (ii) analysis of the long-term physical and chemical stability of BU formulations or (iii) development of combination formulations containing a corticosteroid and an additional drug used for the treatment of asthma and other respiratory disease states, such as but not limited to beta-agonists.

### Example 6

Employing the steps in the above examples, Applicants prepared selected formulations. Table 4 lists the lot and formulation compositions used. Based on the 5% budesonide load, 4 mg of total powder is calculated to deliver a nominal dose of 200 ug of budesonide, which is comparable to the current Pulmicort Turbohaler. Higher drug loads (up to 10%, or 50%, or 90%) require even less total powder to deliver the same nominal budesonide dose. In addition, lower total powder dosages are based on improved delivery efficiency and efficacy.

TABLE 4
Powder Lot and formulation compositions with targeted ratios.

| | Formulation | Atomization | Percent | Scale |
|---|---|---|---|---|
| Lot | (Budesonide/Leu/DPPC) | gas (gr/min) | Yield | (gr) |
| A | 5/90/5 | 18.5 | 48 | 5 |
| B | 50/50/0 | 10 | 60 | 5 |
| C | 50/45/5 | 14 | 72 | 5 |
| D | 90/10/0 | 15 | | 5 |
| E | 10/90/0 | 15 | 44 | 5 |

### Example 7

Several 5 gram scale lots of BU powder were spray-dried for testing. In all cases, the budesonide and DPPC when included in the formulation was dissolved in 700 ml. of ethanol and the leucine was dissolved in 300 ml. of water. The two solutions were then heated, combined using a static mixer together and then spray dried. Atomization gas flow rate was adjusted to the indicated rate in Table 4 such that the size of the particles as measured by an online device was between 6 and 7 microns. See U.S. Ser. No. 10/101,563 (US2003/0180283) “Method and Apparatus for Producing Particles. Yields of each run are shown in Table 4. The spray-drying conditions used are disclosed below in Table 5.

TABLE 5
Spray Drying Conditions for 5 gram lots.

| | |
|---|---|
| Inlet Temp [C.] | 115 |
| Outlet Temp [C.] | 52 |
| Liquid Flow [mL/min] | 60 |
| Drying gas [kg/hr] | 95 |
| Solution Temp [C.] | 50 |

Geometric size, as measured by RODOS at either 1 or 2 bar, FPF and tap density for the 5 gram test lots are given in Table 6.

TABLE 6
Sample measurements

| | | RODOS @ 2 | | | |
|---|---|---|---|---|---|
| | RODOS @ 1 | bar | FPF < 5.6 | FPF < 3.4 | Tap |
| Lot | bar (microns) | (microns) | um | um | density |
| A | 7.08 | 6.65 | 70 | 53 | |
| B | 6.95 | 6.05 | | | .08 |
| C | 7.01 | 5.84 | | | .08 |

The figures illustrate the difference in properties between at least one BU powder formulation and the raw BU.

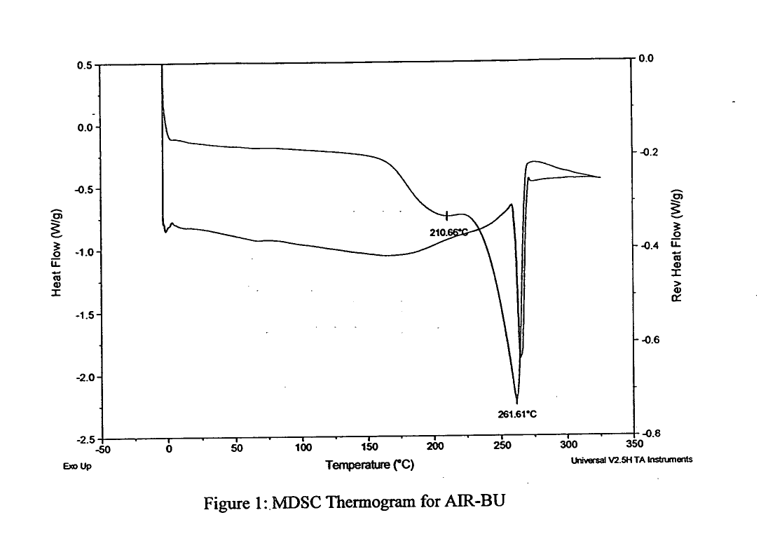

FIG. 1 is a MDSC thermogram for a budesonide formulation of the instant invention. The formulation appears to be predominately crystalline with a budesonide melting point of 266° C. No recrystallization events occurred and there was no evidence of a glass transition in the reversing heat flow for the formulation.

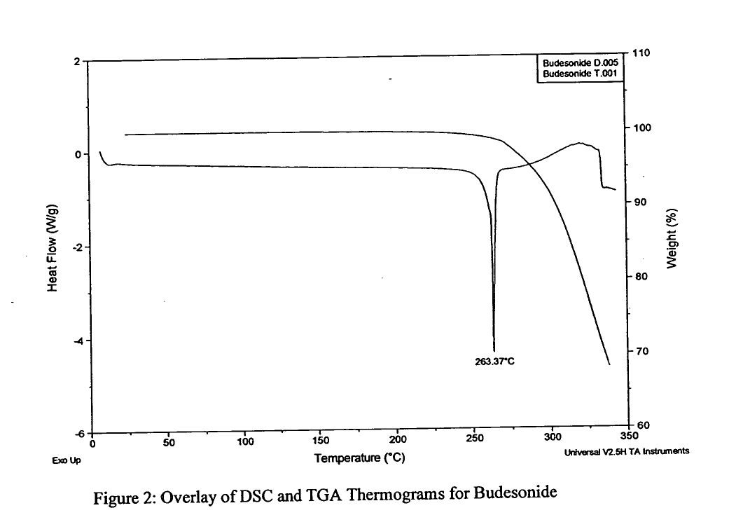

FIG. 2 is an overlay of DSC and TGA thermograms for the raw drug budesonide. Budesonide exhibited a crystalline melting transition at 263° C. This melting transition is verified through the TGA curve which shows a rapid loss in weight beginning at 260° C. No volatile content was detected for budesonide.

### Example 8

Applicants spray-dried a 5% BU powder on a larger scale than outlined above. A sixty-six (66) gram scale, spray drying operation was conducted to produce a 5% BU powder. The formulation contained 5/90/5 w/w budesonide/leucine/DPPC. The yield was about 50 gram of dry powder, which represented an approximately 75% recovery. The spray-drying conditions used can be seen below in Table 7.

TABLE 7
Spray Drying Conditions.

| | |
|---|---|
| Inlet Temp [C.] | 111 |
| Outlet Temp [C.] | 54 |
| Liquid Flow [mL/min] | 60 |
| Drying gas [kg/hr] | 100 |
| Solution Temp [C.] | 50 |
| Chamber Pressure [in H₂O] | −2 |
| Atomization Gas [g/min] | 16 |
| Nozzle Configuration | 67147/2850 |

The following characteristics were observed.

FPF data were collected and the percent (%) total dose less than 5.6 μm was 72.0 (2.0 standard deviation) and the percent (%) total dose less than 3.4 μm was 56.1 (2.8 standard deviation).

Table 7 shows the geometric particle size distribution (gPSD) data. The x₅₀ gPSD ranged from 8.94 μm at 0.5 bar primary pressure to 5.11 μm at 4 bar.

TABLE 7
gPSD dispersibility data (bulk powder).

| Primary | Depression | x₁₀ | | | |
|---|---|---|---|---|---|
| Pressure (bar) | Pressure (bar) | (μm) | x₅₀ (μm) | x₉₀ (μm) | Cₒₚₜ (%) |
| 0.5 | 4 | 1.710 | 8.94 | 24.560 | 7.56% |
| 1 | 7 | 1.570 | 6.64 | 17.060 | 8.27% |
| 2 | 16 | 1.370 | 6.04 | 15.230 | 7.06% |
| 4 | 35 | 1.220 | 5.11 | 13.450 | 6.99% |

While various embodiments of the present invention have been described above, it should be understood that they have been presented by way of example only, and not limitation. For example, the present invention is not limited to the physical arrangements or dimensions illustrated or described. Nor is the present invention limited to any particular design or materials of construction. As such, the breadth and scope of the present invention should not be limited to any of the above-described exemplary embodiments, but should be defined only in accordance with the following claims and their equivalents.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

We claim:

1. A method of treating a corticosteroid-responsive condition of the air passage ways and lungs by delivering a corticosteroid to the pulmonary system of a patient, comprising administering a particle mass of less than 5 mg comprising a corticosteroid from an inhaler wherein at least 50% of the particle mass from the inhaler is delivered to the pulmonary system of the patient.

2. The method of claim 1, wherein the corticosteroid-responsive condition of the air passage ways and lungs (C-RCAL) is selected from the group consisting of asthma, allergic rhinitis, non-allergic rhinitis, non-malignant proliferative diseases of the air passageways and lungs, and inflammatory diseases of the air passageways and lungs.

3. The method of claim 1, wherein the corticosteroid-responsive condition of the air passageways and lungs (C-RCAL) is selected from the group consisting of asthma, chronic bronchitis, inflammatory diseases of the respiratory tract, obstructive diseases of the respiratory tract, chronic obstructive pulmonary disease (COPD), respiratory tract infection and upper respiratory tract disease.

4. The method of claim 1, wherein the corticosteroid is selected from the group consisting of aldosterone, beclomethasone, betamethasone, budesonide, cloprednol, cortisone, cortivazol, deflazacort, deoxycortone, desonide, dexamethasone, difluorocortolone, fluclorolone, fluorocortisone, flumethasone, flunisolide, fluocinolone, fluocinonide, fluorocortolone, fluorometholone, flurandrenolone, halcinonide, hydrocortisone, meprednisone, methylprednisolone, paramethasone, prednisolone, prednisone and triamcinolone, and their respective pharmaceutically acceptable salts or esters.

5. A method of claim 1, wherein the patient has a peak inspiratory flow rate of about 15 liters per minute.

6. A method of claim 1, wherein the inhaler contains less than 4 milligrams of the mass.

7. A method of claim 1, wherein the dose is about 3 milligrams.

8. A method of claim 1, wherein the mass has a tap density of less than about 0.4 g/cm³.

9. A method of claim 1, wherein the mass has a tap density of less than about 0.1 g/cm³.

10. A method of claim 1, wherein the mass has a tap density of less than about 0.05 g/cm³.

11. A method of claim 1, wherein the mass mean geometric diameter of the mass emitted from the inhaler is between about 3 microns and about 15 microns.

12. A method of claim 1, wherein the mass mean geometric diameter of the mass emitted form the inhaler is between about 3 microns and about 10 microns.

13. A method of claim 1, wherein the mass mean aerodynamic diameter of the mass emitted from the inhaler is between about 1 and about 5 microns.

14. A method of claim 1, wherein the mass mean aerodynamic diameter of the mass emitted from the inhaler is between about 1 and about 3 microns.

15. A method of claim 1, wherein the emitted dose from the inhaler is greater than about 70%.

16. A method of claim 1, wherein the emitted dose from the inhaler is greater than about 80%.

17. A method of claim 1, wherein the mass consists essentially of spray-dried particles.

18. A method of claim 1, wherein the patient is in anaphylaxis.

19. A method of claim 1, wherein the patient is asthmatic.

20. A method of claim 1, wherein the patient is a child.

21. A method of treating corticosteroid-responsive condition of the air passageways and lungs (C-RCAL) by delivering a budesonide to the pulmonary system of a compromised patient, comprising administering a particle mass of less than 5 mg comprising budesonide from an inhaler wherein at least 50% of the particle mass from the inhaler is delivered to the pulmonary system of the patient.

22. The method of claim 21, wherein the particle mass is less than 4 mg.

23. The method of claim 22 further comprising a nominal dose of about 100 to about 350 ug of budesonide.

24. The method of claim 23, wherein the nominal dose of budesonide is about 200 ug.

25. The method of claim 21, wherein the particle mass comprises 5/90/5 weight percent of budesonide/leucine/DPPC.
